(19) United States
(12) Patent Application Publication
(10) Pub. No.: US 2022/0218937 A1
(43) Pub. Date: Jul. 14, 2022

(54) HUMIDIFIER

(71) Applicant: Murata Manufacturing Co., Ltd., Kyoto (JP)

(72) Inventor: Yuzo HIGASHIYAMA, Kyoto (JP)

(21) Appl. No.: 17/657,375

(22) Filed: Mar. 31, 2022

Related U.S. Application Data
(63) Continuation of application No. PCT/JP2020/033978, filed on Sep. 8, 2020.

(30) Foreign Application Priority Data
Oct. 17, 2019 (JP) 2019-190178

(51) Int. Cl. A61M 16/16; A61M 16/08; A61M 16/10

(52) U.S. Cl. CPC A61M 16/16; A61M 16/0808; A61M 2205/123; A61M 2205/42; A61M 16/109

(57) ABSTRACT

A humidifier is provided with a water storage tank, a second housing to which the tank is detachably mounted, and a heater for vaporizing water stored in the tank. The tank is configured with a tank body having a space therein, and a partition wall rising from an inner surface of the tank body. A first tank hole that causes an inside and an outside of the tank body to communicate with each other opens in the tank body. The partition wall partitions the space inside the tank body into a water storage space for storing water, and a trap space. The trap space communicates with an outside of the tank via the first tank hole, and communicates with the water storage space.

### CROSS REFERENCE TO RELATED APPLICATION

This is a continuation of International Application No. PCT/JP2020/033978 filed on Sep. 8, 2020, which claims priority from Japanese Patent Application No. 2019-190178 filed on Oct. 17, 2019. The contents of these applications are incorporated herein by reference in their entireties.

### BACKGROUND ART

### Technical Field

The present disclosure relates to a humidifier.

A gas supply device described in Patent Document 1 is provided with a blower and a humidifier. In this gas supply device, air force-fed from the blower enters the humidifier, and is humidified in the humidifier, and then the humidified air is sent to a user. The humidifier is provided with a humidifier body, a water storage tank mounted to the humidifier body, and a heater for vaporizing water stored in the tank. Then, air is sent by the blower, via a tank hole that causes the inside and the outside of the tank to communicate with each other.
Patent Document 1: Japanese Unexamined Patent Application Publication No. 2011-045730

### BRIEF SUMMARY

In a humidifier as described in Patent Document 1, a tank may be inclined when a humidifier is used. When the tank is inclined, there is a possibility that water stored in the tank leaks outside the tank via a tank hole.

In order to solve the problem described above, an aspect of the present disclosure is a humidifier including a water storage tank, a humidifier body to which the tank is detachably mounted, and a humidification promoting mechanism for vaporizing water stored in the tank, in which a gas humidified by the humidification promoting mechanism is discharged from the tank, wherein the tank includes a tank body having a space therein, and a partition wall rising from an inner surface of the tank body, a tank hole causing an inside and an outside of the tank body to communicate with each other is provided in the tank body, the partition wall partitions a space inside the tank body into a water storage space for storing water, and a trap space, the trap space communicates with an outside of the tank via the tank hole, and the water storage space communicates with the trap space.

According to the above configuration, the water storage space communicates with the tank hole via the trap space. Thus, in order to reach the tank hole, water stored in the water storage space is to pass through the trap space. Since water can be stored in this trap space, even when the water stored in the water storage space flows out from the water storage space, it is possible to suppress direct leakage from the tank hole.

In a humidifier, it is possible to suppress leakage of water stored in a tank outside the tank.

### BRIEF DESCRIPTION OF THE DRAWINGS

FIG. 1 is a perspective view illustrating a main body unit and a base unit of a CPAP device.

FIG. 2 is a perspective view illustrating the main body unit and the base unit of the CPAP device in a first use state.

FIG. 3 is an explanatory diagram illustrating a schematic configuration of the CPAP device in the first use state.

FIG. 4 is an explanatory diagram illustrating a schematic configuration of the CPAP device in a second use state.

FIG. 5 is a perspective view in which the base unit is viewed from an angle different from that of FIG. 1.

FIG. 6 is an end view of the base unit (end view taken along a line 6-6 in FIG. 7).

FIG. 7 is an end view of the base unit taken along a line 7-7 in FIG. 6.

FIG. 8 is a schematic view illustrating the first use state of the CPAP device.

FIG. 9 is a schematic view illustrating the second use state of the CPAP device.

FIG. 10 is an end view of a base unit in a second embodiment (end view taken along a line 10-10 in FIG. 11).

FIG. 11 is an end view of the base unit taken along a line 11-11 in FIG. 10.

FIG. 12 is an end view of a base unit in a modification.

FIG. 13 is an end view of a base unit in a modification.

### DETAILED DESCRIPTION

Hereinafter, an embodiment in which a humidifier is applied to a continuous positive airway pressure (CPAP) device will be described with reference to the figures.

### First Embodiment

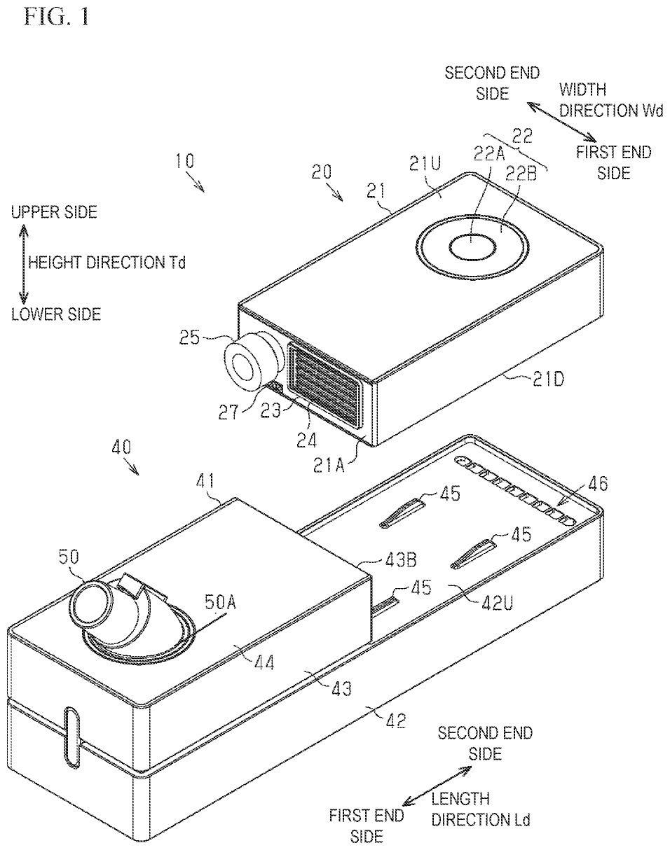

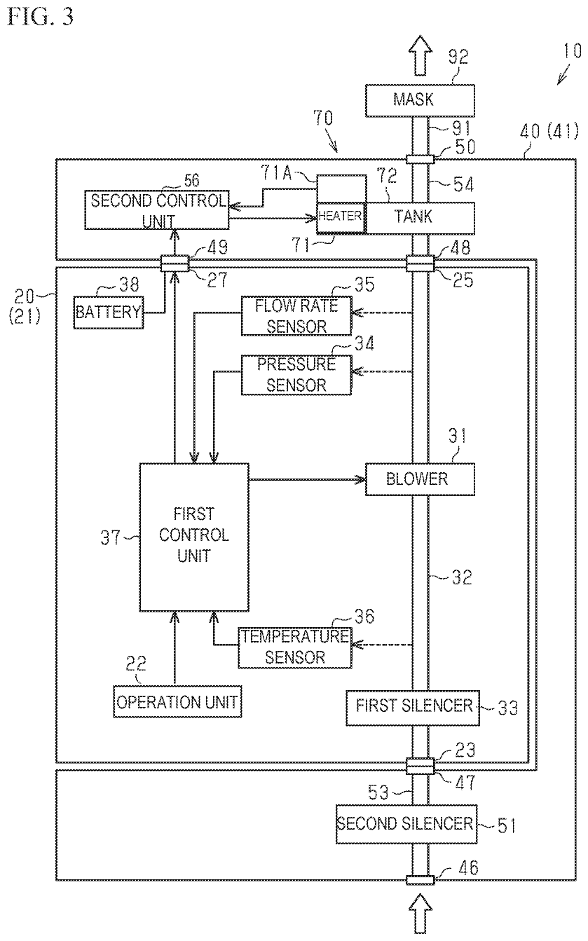

First, a configuration of a CPAP device that sends air introduced into the device to a user's respiratory tract will be described. As illustrated in FIG. 1, a CPAP device 10 is provided with a main body unit 20 and a base unit 40. Additionally, as illustrated in FIG. 3, the main body unit 20 is provided with a blower 31 as a main constituent element. The base unit 40 is provided with a second silencer 51 and a humidifier 70 as main constituent elements.

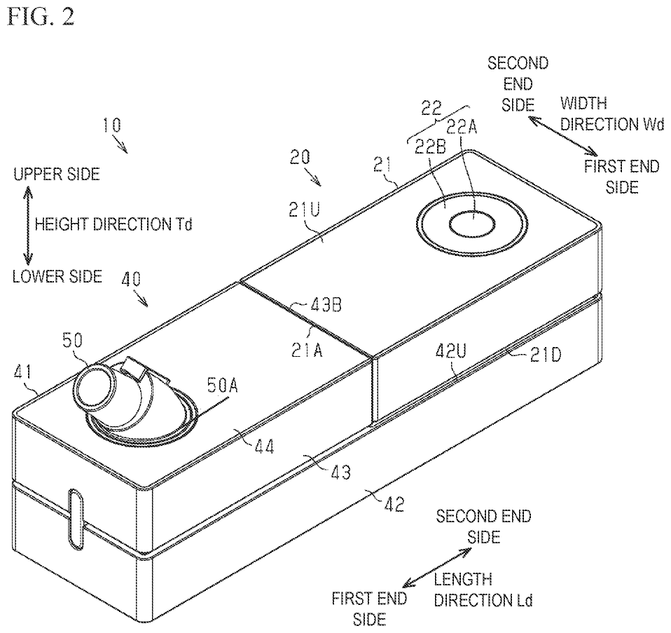

As illustrated in FIG. 1 and FIG. 2, the main body unit 20 can be attached to and detached from the base unit 40. In the present embodiment, the CPAP device 10 is configured to be usable in a first use state and a second use state. The first use state is a state in which the main body unit 20 is loaded into the base unit 40 and used, and the second use state is a state in which the main body unit 20 is used without being loaded into the base unit 40. In other words, in the first use state, as illustrated in FIG. 3, the main body unit 20 and the base unit 40 are used. As illustrated in FIG. 4, in the second use state, only the main body unit 20 is used, and the base unit 40 is not used.

Next, a configuration of the main body unit 20 will be described.

As illustrated in FIG. 1, the main body unit 20 is provided with a first housing 21 in a flat rectangular parallelepiped shape. As illustrated in FIG. 4, the blower 31 and the like are built inside the first housing 21. Note that, in the following description, when directions related to the first housing 21 are denoted, as illustrated in FIG. 1, a thickness direction of the first housing 21 is defined as a height direction Td. In addition, a long side direction of the first housing 21 is defined as a length direction Ld, and a short side direction is defined as a width direction Wd.

As illustrated in FIG. 1, an operation unit 22 for operating the main body unit 20 is provided on an upper side surface 21U of the first housing 21. In this embodiment, the operation unit 22 is configured with a switch 22A in a circular shape, and a switch 22B in an annular shape arranged so as to surround the switch 22A. Both the switches 22A and 22B are push button switches, and operating these switches makes it possible to turn on or off a power supply of the main body unit 20, change settings, and the like.

In a first end surface 21A which is an end surface on a first end side in the length direction Ld of the first housing 21, a first introduction port 23 for introducing air from the outside to the inside of the first housing 21 opens. A filter 24 that filters dust and the like contained in the air introduced into the first housing 21 is mounted to the first introduction port 23.

As illustrated in FIG. 4, a main flow path 32 through which air flows is defined inside the first housing 21 of the main body unit 20. In the main body unit 20, an upstream end of the main flow path 32 is connected to the first introduction port 23. The blower 31 that sends out air from the first introduction port 23 to a downstream side is mounted in midstream of the main flow path 32. The blower 31 is, for example, a centrifugal fan. In the main flow path 32, a first silencer 33 is mounted between the first introduction port 23 and the blower 31. The first silencer 33 attenuates a flow sound of air that flows through the main flow path 32 along with driving of the blower 31.

A pressure sensor 34 that detects pressure of air downstream of the blower 31 in the main flow path 32 is mounted inside the first housing 21. Additionally, a flow rate sensor 35 that detects a flow rate of air downstream of the blower 31 in the main flow path 32 is mounted inside the first housing 21. Further, a temperature sensor 36 that detects a temperature of air flowing through the main flow path 32 is mounted inside the first housing 21. A first lead-out portion 25 for leading out air from the inside to the outside of the first housing 21 is connected to a downstream end of the main flow path 32.

As illustrated in FIG. 1, the first lead-out portion 25 protrudes from the first end surface 21A of the first housing 21. The first lead-out portion 25 is arranged so as to be aligned with the first introduction port 23 in the width direction Wd of the first housing 21. The first lead-out portion 25 has a cylindrical shape as a whole, and protrudes from the first end surface 21A along the length direction Ld. Then, an internal space of the first lead-out portion 25 communicates with the main flow path 32 inside the first housing 21.

In the first end surface 21A, a first connector 27 for electrically connecting the main body unit 20 to the base unit 40 is recessed. The first connector 27 is a so-called female connector, and a plurality of terminals is provided therein. The first connector 27 is arranged on a lower side of the first lead-out portion 25.

Next, an electrical configuration of the main body unit 20 of the CPAP device 10 will be described.

As illustrated in FIG. 4, the main body unit 20 is provided with a first control unit 37 for controlling operation of the blower 31. Note that, the first control unit 37 is electrically connected to the first connector 27 by wiring (not illustrated).

The first control unit 37 may be configured as circuitry including 1) one or more processors that execute various processes in accordance with a computer program (software), 2) one or more dedicated hardware circuits, such as an application-specific integrated circuit (ASIC), that execute at least some processes of the various processes, or 3) a combination thereof. The processor includes a CPU and memories, such as a RAM and a ROM, and the memory stores program codes or directives configured to cause the CPU to execute the processes. The memory or a computer-readable medium includes any available medium that can be accessed from a general purpose or dedicated computer.

In the first housing 21 of the main body unit 20, a battery 38 is provided for supplying power to the blower 31, the pressure sensor 34, the flow rate sensor 35, the temperature sensor 36, and the first control unit 37. The battery 38 is a secondary battery that can be repeatedly charged, and is charged by connecting a charging cable (not illustrated) to the main body unit 20. Further, the battery 38 is also electrically connected to the first connector 27.

The first control unit 37 is inputted with a signal indicating an operation from the operation unit 22. The first control unit 37 is inputted with a pressure value detected by the pressure sensor 34. The first control unit 37 is inputted with a flow rate value detected by the flow rate sensor 35. The first control unit 37 is inputted with a temperature value detected by the temperature sensor 36. The first control unit 37 is configured to, based on these values inputted, increase or decrease the number of rotations of the blower 31 by control such as feedback control or feed-forward control, thereby controlling an amount of air feeding and the like. For example, the first control unit 37 determines an exhalation state of a user based on values detected by the pressure sensor 34 and the flow rate sensor 35, and controls a pressure value of air to be fed to the user so as to be synchronized with the exhalation state. Further, the first control unit 37 controls power supply from the battery 38 to the first connector 27.

Next, structure of the base unit 40 will be described.

As illustrated in FIG. 1, the base unit 40 is provided with a second housing 41 in an L-shape in side view. The second housing 41 is roughly divided into a base housing 42 in a flat rectangular parallelepiped shape, and a protruding housing 43 in a flat rectangular parallelepiped shape positioned on an upper side of the base housing 42.

A dimension in a longitudinal direction of the base housing 42 is larger than a dimension in the length direction Ld of the first housing 21. A dimension in a short direction of the base housing 42 is the same as a dimension in the width direction Wd of the first housing 21. Note that, in the following description, the longitudinal direction of the base housing 42 of the second housing 41 is along the length direction Ld of the first housing 21, and the short direction of the base housing 42 is along the width direction Wd of the first housing 21.

The protruding housing 43 protrudes from an upper surface on a first end side in the length direction Ld of the base housing 42. An end on a first end side in the length direction Ld of the protruding housing 43 coincides with the end on the first end side in the length direction Ld of the base housing 42. A dimension in the height direction Td of the protruding housing 43 is substantially the same as a dimension in the height direction Td of the first housing 21. A dimension in the width direction Wd of the protruding housing 43 is substantially the same as a dimension in the width direction Wd of the first housing 21. A dimension in the length direction Ld of the protruding housing 43 is a value obtained by subtracting the dimension in the length direction Ld of the first housing 21 from the dimension in the longitudinal direction of the base housing 42.

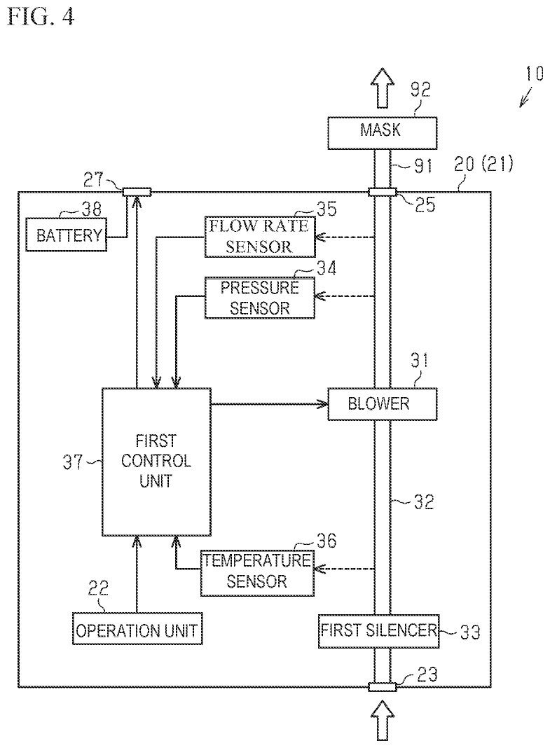

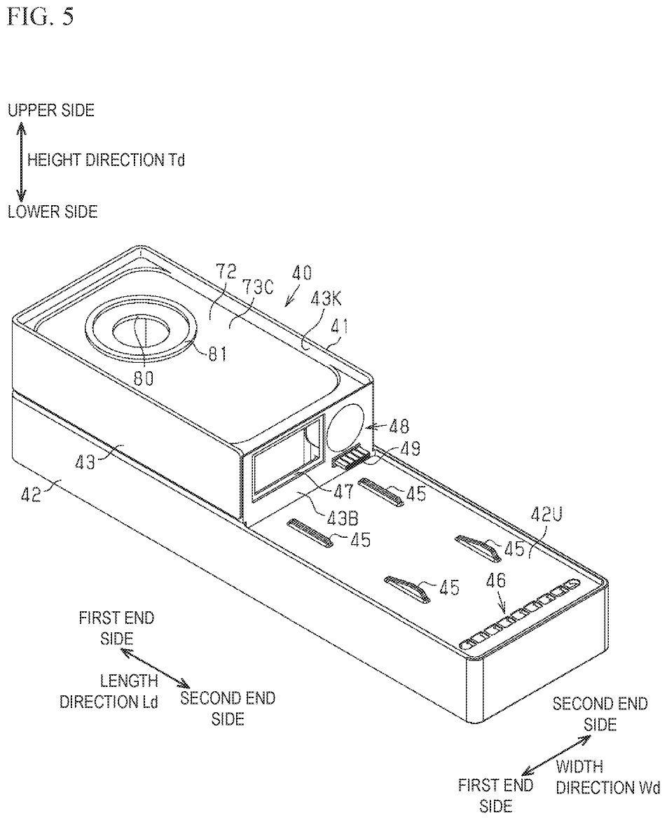

The base housing 42 and the protruding housing 43 both have a box shape having a cavity therein. Further, an internal space of the base housing 42 and an internal space of the protruding housing 43 are continuous with each other. A wall portion of the protruding housing 43 on a side opposite to the base housing 42, that is, a wall portion on an upper side is configured as a lid 44 that can be opened and closed. The lid 44 is removable, and with the lid 44 of the protruding housing 43 removed, the internal space of the protruding housing 43 and a part of the internal space of the base housing 42 are exposed. Note that, in FIG. 5, the base unit 40 is illustrated with the lid 44 removed.

As illustrated in FIG. 5, an upper side surface 42U of the base housing 42 is provided with a protrusion 45 protruding toward an upper side in the height direction Td of the base housing 42. In this embodiment, two protrusions 45 are provided for each row along the length direction Ld of the base housing 42. Then, two rows of the protrusions 45 are provided in the width direction Wd of the base housing 42. That is, in total, four protrusions 45 are provided.

In the upper side surface 42U of the base housing 42, a second introduction port 46 for introducing air from the outside to the inside of the base housing 42 opens. In the present embodiment, the second introduction ports 46 is plurally provided. The second introduction ports 46 are arranged so as to be aligned over substantially an entire region in the width direction Wd of the base housing 42. Further, each of the second introduction ports 46 is arranged in a vicinity of an edge on a second end side in the length direction Ld of the upper side surface 42U of the base housing 42. Note that, the upper side surface 42U of the base housing 42 functions as a surface for placing the main body unit 20.

As illustrated in FIG. 3, an upstream side flow path 53 through which air pulled into the blower 31 of the main body unit 20 flows is defined inside the second housing 41 of the base unit 40. In the base unit 40, an upstream end of the upstream side flow path 53 is connected to the second introduction port 46.

The second silencer 51 is mounted in midstream of the upstream side flow path 53. The second silencer 51 attenuates a flow sound of air flowing through the upstream side flow path 53. Note that, a size of volume of the second silencer 51 is larger than volume of the first silencer 33 of the main body unit 20, and a sound attenuation effect is higher than that of the first silencer 33 of the main body unit 20.

A downstream end of the upstream side flow path 53 is connected to a second lead-out port 47 for leading out air from the inside to the outside of the second housing 41. As illustrated in FIG. 5, the second lead-out port 47 opens in a surface connected to the upper side surface 42U of the base housing 42 of respective end surfaces on both sides in the length direction Ld of the protruding housing 43, that is, in a second end surface 43B which is a side surface on a second end side in the length direction Ld in the protruding housing 43. An opening shape of the second lead-out port 47 is the same as an opening shape of the first introduction port 23 of the main body unit 20.

Additionally, as illustrated in FIG. 3, a downstream side flow path 54 through which air fed from the blower 31 of the main body unit 20 flows is defined inside the second housing 41 of the base unit 40. A tank 72 of the humidifier 70 is mounted in midstream of the downstream side flow path 54. The humidifier 70 is provided with the tank 72 described above, a heater 71, and a heater temperature sensor 71A. The tank 72 is configured to be capable of being attached to and detached from the second housing 41, and is capable of storing water therein. Air introduced inside the humidifier 70 is led out from the humidifier 70 through the tank 72, thereby humidifying the air. The heater 71 heats water in the tank 72. The heater temperature sensor 71A detects a temperature of the heater 71.

As illustrated in FIG. 5, a third introduction hole 48 for introducing air from the outside to the inside of the protruding housing 43 opens in the second end surface 43B of the protruding housing 43 in the base unit 40. The third introduction hole 48 is arranged so as to be aligned with the second lead-out port 47 in the width direction Wd in the protruding housing 43. The third introduction hole 48 has a circular shape in plan view, and an outer diameter of the third introduction hole 48 is larger than an outer diameter of the first lead-out portion 25 in the first housing 21.

In the second end surface 43B, a second connector 49 for electrically connecting the main body unit 20 to the base unit 40 protrudes. The second connector 49 is a so-called male connector corresponding to a shape of the first connector 27 described above, and is provided with a plurality of terminals therein. The second connector 49 is arranged on a lower side of the third introduction hole 48.

As illustrated in FIG. 1, a cylindrical third lead-out portion 50 for leading out air from the inside to the outside of the second housing 41 protrudes from the lid 44 of the second housing 41. A central axis line of the third lead-out portion 50 is inclined with respect to the height direction Td in the protruding housing 43. An internal space of the third lead-out portion 50 communicates with the downstream side flow path 54.

Next, an electrical configuration of the CPAP device 10 in the base unit 40 will be described.

As illustrated in FIG. 3, the base unit 40 is provided with a second control unit 56 that controls operation of the heater 71. The second control unit 56 may be configured as circuitry including 1) one or more processors that execute various processes in accordance with a computer program (software), 2) one or more dedicated hardware circuits, such as an application-specific integrated circuit (ASIC), that execute at least some processes of the various processes, or 3) a combination thereof. The processor includes a CPU and memories, such as a RAM and a ROM, and the memory stores program codes or directives configured to cause the CPU to execute the processes. The memory or a computer-readable medium includes any available medium that can be accessed from a general purpose or dedicated computer.

Electric power is supplied to the second control unit 56 from the battery 38 of the main body unit 20 with the first connector 27 of the main body unit 20, and the second connector 49 interposed therebetween. Additionally, a signal indicating a temperature value of air detected by the temperature sensor 36 is inputted from the first control unit 37 to the second control unit 56 with the first connector 27 of the main body unit 20, and the second connector 49 interposed therebetween.

The second control unit 56 sets a target heater temperature for heating water in the tank 72, based on the inputted air temperature value. For example, the second control unit 56 sets the target heater temperature with a predetermined calculation formula. Then, the second control unit 56 is configured to drive the heater 71, based on a heater temperature detected by the heater temperature sensor 71A so as to set the heater temperature to the target heater temperature by control such as feedback control or feed-forward control. The second control unit 56 adjusts a water temperature in the tank 72 by the heater 71. Then, when the heater temperature reaches the target heater temperature, the second control unit 56 controls the heater 71 so as to maintain the heater temperature at the target heater temperature.

Next, internal structure of the base unit 40, particularly the humidifier 70, will be described in detail.

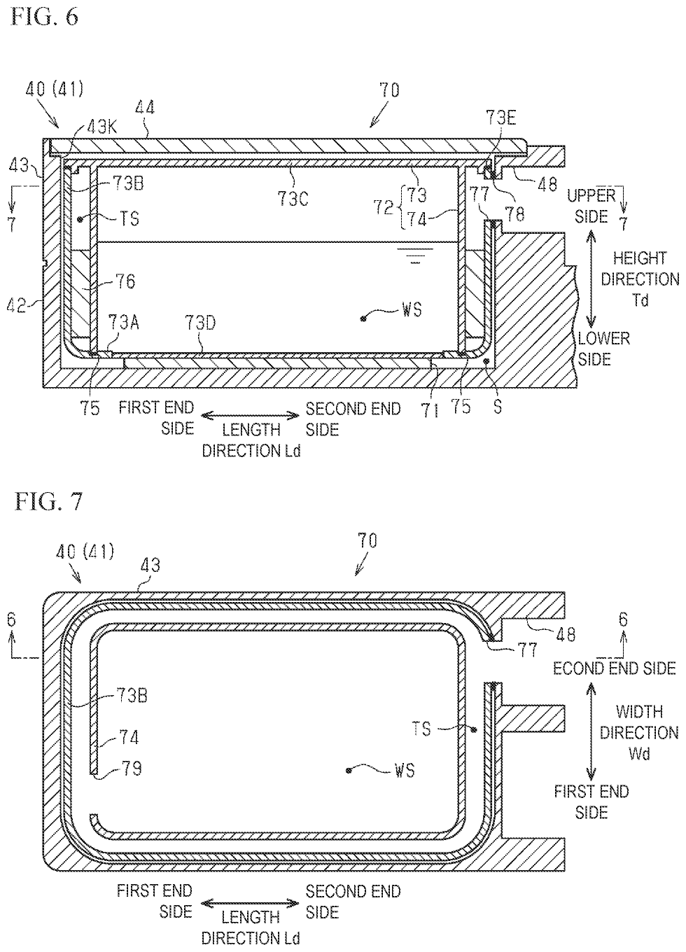

As illustrated in FIG. 6, a housing space S in a rectangular parallelepiped shape is defined by the internal space of the base housing 42 and the internal space of the protruding housing 43. With the lid 44 of the base unit 40 removed, an upper side of the protruding housing 43 opens as an attachment/detachment port 43K. The heater 71 in a plate shape is arranged on a part of the base unit 40 on a side opposite to the attachment/detachment port 43K, that is, on a bottom of the base housing 42, which is on a lower side in the height direction Td. In this embodiment, the heater 71 is mounted on a bottom surface of the base housing 42.

In the housing space S, the water storage tank 72 can be housed on an upper side in the height direction Td of the heater 71. The tank 72 has a substantially rectangular parallelepiped box shape. Water stored in the tank 72 is heated by the heater 71, and vaporization is promoted. In the present embodiment, the second housing 41 functions as a humidifier body, and the heater 71 functions as a humidification promoting mechanism.

As illustrated in FIG. 6, the tank 72 is configured with a tank body 73 having a space therein, and a partition wall 74 rising from an inner surface of the tank body 73. In the tank body 73, a bottom wall 73A has a flat plate shape, and has a rectangular shape when viewed in plan (viewed in a direction perpendicular to a main surface of the lid 44). When the humidifier 70 is viewed from above (viewed in a direction perpendicular to a main surface of the lid 44), a central portion of the bottom wall 73A is a heater contact portion 73D facing the heater 71. Further, a material for the heater contact portion 73D is metal. As described above, since most of the bottom wall 73A is made of metal, thermal conductivity of the bottom wall 73A is relatively high. In addition, in the bottom wall 73A, a groove-shaped recessed portion 75 is recessed so as to surround the heater contact portion 73D.

In the tank body 73, a side wall 73B extends upward in the height direction Td from an edge of the bottom wall 73A. The side wall 73B extends from entire four sides of the bottom wall 73A, and has a cylindrical shape as a whole. A material for the side wall 73B is resin. Thus, thermal conductivity of the side wall 73B is lower than that of the bottom wall 73A, which is mostly made of metal.

An upper wall 73C in a rectangular shape in plan view is mounted to an upper side in the height direction Td of the side wall 73B. The upper wall 73C covers a space surrounded by the side wall 73B from above. A material for the upper wall 73C is resin. In this manner, the tank body 73 is configured with the bottom wall 73A, the side wall 73B, and the upper wall 73C, and has a rectangular parallelepiped box shape as a whole.

The partition wall 74 in a rectangular cylindrical shape extends downward in the height direction Td from an inner surface, which is a surface of the upper wall 73C on an internal space side of the tank body 73. A lower end of the partition wall 74 is fitted into the recessed portion 75. Note that, a contact portion between the partition wall 74 and the recessed portion 75 is sealed by an elastically deformable seal (not illustrated). A material for the partition wall 74 is resin. That is, the material for the partition wall 74 is a material having lower heat conductivity than that of the bottom wall 73A including the heater contact portion 73D.

The partition wall 74 is an integrally molded product with the upper wall 73C. Further, the upper wall 73C and the partition wall 74 can be attached to and detached from the bottom wall 73A and the side wall 73B. To be specific, the upper wall 73C and the partition wall 74 can be attached to and detached from the bottom wall 73A and the side wall 73B, by inserting and pulling the lower end of the partition wall 74 into and from the recessed portion 75. Further, an elastically deformable seal member 73E is mounted to a connection portion between the upper wall 73C and the side wall 73B. The seal member 73E seals between the upper wall 73C and the side wall 73B. The seal member 73E has an annular shape, and is mounted to an entire upper end of the side wall 73B.

The partition wall 74 partitions the internal space of the tank body 73 into a water storage space WS for storing water, and a trap space TS. When viewed from above, a space inside the partition wall 74 is the water storage space WS. Further, a space outside the partition wall 74 and inside the side wall 73B is the trap space TS. The trap space TS is sealed by the tank body 73 and the partition wall 74. Note that, when the humidifier 70 is used, water is stored in the water storage space WS.

A sponge 76 is housed in the trap space TS as a water absorbing member for absorbing water. The sponge 76 is arranged over an entirety of the trap space TS when viewed from above. Further, an upper end in the height direction Td of the sponge 76 is positioned substantially at a center in the height direction Td of the trap space TS, and a lower end in the height direction Td of the sponge 76 is positioned slightly above in the height direction Td than the bottom wall 73A.

A first tank hole 77 in a circular shape in plan view opens at a portion facing the third introduction hole 48 in a wall portion on a second end side in the length direction Ld of the side wall 73B of the tank body 73. The first tank hole 77 causes the trap space TS in the internal space of the tank body 73 and a space between the tank body 73 and the second housing 41 to communicate with each other. The first tank hole 77 opens toward the second end side in the length direction Ld when viewed from the inside of the tank body 73. That is, a central axis line of the first tank hole 77 is parallel to the length direction Ld. In the present embodiment, the first tank hole 77 functions as a tank hole.

Additionally, a first O-ring 78 in an annular shape is arranged between an outer surface on the second end side in the length direction Ld of the side wall 73B of the tank body 73, and a surface on a side of the housing space S of the second housing 41. A flow path of the downstream side flow path 54 that leads to an internal space of the tank 72 through the third introduction hole 48 via the first tank hole 77, and the housing space S are separated from each other by the first O-ring 78.

As illustrated in FIG. 7, a partition wall hole 79 in a circular shape in plan view opens in a wall portion on a first end side in the length direction Ld of the partition wall 74. The partition wall hole 79 causes the water storage space WS and the trap space TS to communicate with each other. The partition wall hole 79 is positioned closer to the first end side than a center of the tank body 73 in the width direction Wd, and is positioned at the same position as that of the first tank hole 77 in the height direction Td. Thus, when viewed from the inside of the tank body 73, the partition wall hole 79 opens toward the first end side in the length direction Ld on an opposite side to the first tank hole 77. That is, the central axis line of the partition wall hole 79 is parallel to the length direction Ld.

As illustrated in FIG. 5, a second tank hole 80 in a circular shape in plan view opens in the upper wall 73C of the tank 72. The second tank hole 80 causes the inside and the outside of the tank 72 to communicate with each other. The second tank hole 80 is, with the lid 44 closed, arranged at a position facing a lid hole 50A which is an opening on a lid side of the third lead-out portion 50 of the lid 44 illustrated in FIG. 1.

As illustrated in FIG. 5, a second O-ring 81 in an annular shape is arranged on an upper side in the height direction Td of the tank 72 so as to surround an opening edge of the second tank hole 80. With the lid 44 mounted, the second O-ring 81 is sandwiched between the upper wall 73C and the lid 44. Accordingly, a flow path of the downstream side flow path 54 that leads to the inside of the tank 72 through the lid hole 50A and the second tank hole 80, and the housing space S are separated from each other by the second O-ring 81.

Next, an action of the CPAP device 10 in the first use state will be described.

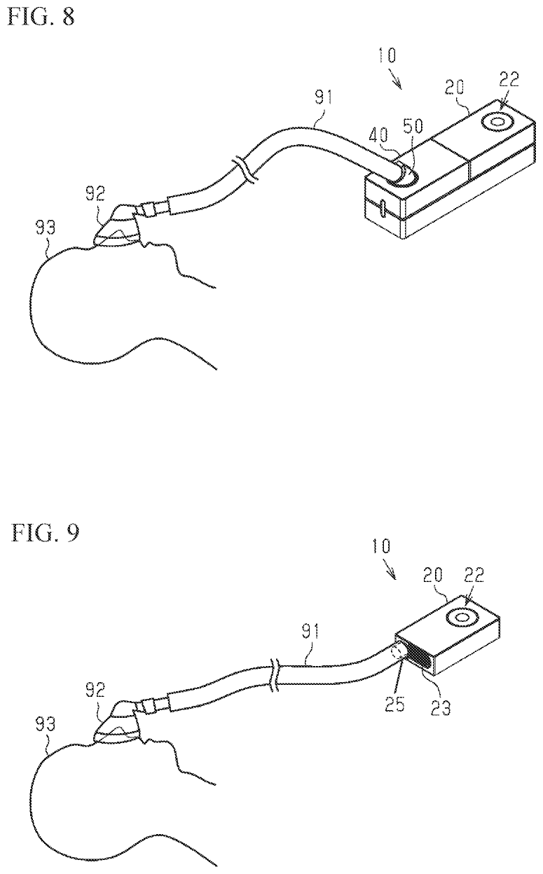

As illustrated in FIG. 8, in the first use state, the main body unit 20 is in a state of being loaded into the base unit 40. To be specific, as illustrated in FIG. 2, the main body unit 20 is placed on the base unit 40 such that a lower side surface 21D in the first housing 21 faces the upper side surface 42U of the base housing 42 in the second housing 41. Further, the first end surface 21A in the first housing 21 and the second end surface 43B of the protruding housing 43 in the second housing 41 face and are in contact with each other. Thus, in the first use state, the CPAP device 10 has an elongated thin rectangular parallelepiped shape as a whole. Note that, in the present embodiment, in the first use state in which the base unit 40 is used to use the CPAP device 10, the protruding housing 43 is used in a direction in which the protruding housing 43 is positioned on an upper side in the vertical direction of the base housing 42.

As illustrated in FIG. 3, in the first use state, the first introduction port 23 of the first housing 21 is connected to the second lead-out port 47 of the second housing 41, and the upstream end of the main flow path 32 of the main body unit 20 is connected to the downstream end of the upstream side flow path 53 of the base unit 40 with these first introduction port 23 and second lead-out port 47 interposed therebetween. In addition, the first lead-out portion 25 of the first housing 21 is inserted into the third introduction hole 48 of the second housing 41, and the downstream end of the main flow path 32 of the main body unit 20 is connected to an upstream end of the downstream side flow path 54 of the base unit 40 with these first lead-out portion 25 and third introduction hole 48 interposed therebetween.

As illustrated in FIG. 8, in the first use state, a first end portion of an air tube 91 is connected to the third lead-out portion 50 of the base unit 40, and a second end portion of the air tube 91 is connected to a mask 92. The mask 92 is worn, for example, so as to cover a nose or mouth of a user 93.

In the first use state of the CPAP device 10, when the operation unit 22 of the main body unit 20 is operated and the power supply of the main body unit 20 is turned on, the blower 31 is driven. Here, due to the protrusion 45 provided on the upper side surface 42U of the base housing 42 of the base unit 40, a gap is generated between the lower side surface 21D of the first housing 21 and the second introduction port 46. Thus, air is pulled inside the CPAP device 10 from this gap through the second introduction port 46. The air pulled inside the CPAP device 10 flows into the humidifier 70 housed in the second housing 41, through the upstream side flow path 53 of the second housing 41, the main flow path 32 of the first housing 21, and the downstream side flow path 54 of the second housing 41. Further, water in the tank 72 is vaporized by being heated by the heater 71. Thus, air flowing into the tank 72 is discharged outside from the third lead-out portion 50 of the second housing 41 together with a water vapor in the tank 72. Thereby, humidified air is fed through the air tube 91 and the mask 92 into a respiratory tract of the user 93.

Next, the CPAP device in the second use state will be described.

As illustrated in FIG. 9, in the second use state, the main body unit 20 is not loaded into the base unit 40, that is, the CPAP device 10 is used only with the main unit 20. In this case, the first end portion of the air tube 91 is connected to the first lead-out portion 25 of the main body unit 20, and the second end portion of the air tube 91 is connected to the mask 92. The mask 92 is worn, for example, so as to cover the nose or mouth of the user 93. In the present embodiment, the humidifier 70 does not function in the second use state.

Next, effects of the humidifier 70 in the above first embodiment will be described.

(1) When the CPAP device 10 is used in the first use state, and the user 93 turns over, or the like, for example, the CPAP device 10 is pulled via the air tube 91, and the CPAP device 10 is inclined. Then, the tank 72 in the CPAP device 10 is also inclined, and a water surface of water stored in the water storage space WS of the tank 72 is also inclined with respect to the tank 72.

Here, it is assumed that the partition wall 74 is not provided, and water is stored in an entirety of the internal space of the tank 72. When the water surface in the tank 72 reaches the first tank hole 77 due to the inclination of the CPAP device 10 as a whole, the water leaks to the third introduction hole 48 via the first O-ring 78. Then, there is a possibility that the leaked water flows into the main flow path 32 of the main body unit 20 and reaches the blower 31.

Further, when the tank 72 is in a state of being inclined and housed, and the water surface in the tank 72 reaches the first tank hole 77, the water leaks to a space between the tank 72 and the second housing 41. Then, there is a possibility that the leaked water reaches the heater 71 and exerts an adverse effect on the heater 71.

In this regard, according to the first embodiment, the internal space of the tank 72 is partitioned into the water storage space WS and the trap space TS by the partition wall 74. Thus, in order to reach the first tank hole 77, water stored in the water storage space WS is to pass through the trap space TS. Since water can be stored in this trap space TS, even when water stored in the water storage space WS flows out from the water storage space WS, it is possible to suppress direct leakage from the first tank hole 77.

(2) According to the first embodiment, the sponge 76 functioning as a water absorbing member is housed in the trap space TS. As described above, when water stored in the water storage space WS flows into the trap space TS via the partition wall hole 79, the sponge 76 absorbs the water. Thus, the water trapped is easily held in the trap space TS, and leakage from the tank 72 via the trap space TS and the first tank hole 77 can be suppressed.

(3) According to the above first embodiment, the first tank hole 77 opens toward the second end side in the length direction Ld. Further, the partition wall hole 79 opens toward the first end side in the length direction Ld. That is, the first tank hole 77 and the partition wall hole 79 open in opposite directions, when viewed from the inside of the tank 72. Then, as described above, water stored in the water storage space WS needs to pass through the trap space TS in order to leak out from the first tank hole 77. Thus, the water stored in the water storage space WS does not leak from the first tank hole 77, unless the second housing 41 housing the tank 72 is inclined such that the first end side in the length direction Ld goes to a lower side, and subsequently the second housing 41 is inclined such that the second end side in the length direction Ld goes to the lower side. As described above, unless a rare situation in which the second housing 41 is inclined in different directions occurs, water does not leak from the first tank hole 77 of the tank 72.

(4) According to the first embodiment, the partition wall 74 and the upper wall 73C can be attached to and detached from the bottom wall 73A and the side wall 73B. Thus, it is possible to replace the partition wall 74 and the upper wall 73C, or to remove and clean the partition wall 74 and the upper wall 73C. Further, by removing the partition wall 74 and the upper wall 73C, the sponge 76 can be easily removed.

(5) According to the first embodiment, the partition wall 74 is the integrally molded product with the upper wall 73C. With this configuration, the tank 72 can be assembled, by simply mounting the partition wall 74 and the upper wall 73C, which form one component, to the bottom wall 73A and the side wall 73B.

(6) Since the upper wall 73C and the partition wall 74 are made as the integrally molded product, and are configured to able to be attached to and detached from the bottom wall 73A and the side wall 73B, a configuration in which air does not leak from between the upper wall 73C and the side wall 73B is required. According to the above first embodiment, the seal member 73E that seals between the upper wall 73C and the side wall 73B is mounted between the upper wall 73C and the side wall 73B. Thus, a configuration in which air does not leak from the trap space TS is realized with a relatively simple configuration.

(7) According to the above first embodiment, in the first use state, the air tube 91 is mounted to the third lead-out portion 50 of the lid 44. By opening and closing the lid 44, the tank 72 can be taken out from the attachment/detachment port 43K. At this time, the air tube 91 can be kept mounted to the lid 44. Thus, without removing the air tube 91, the tank 72 can be attached and detached, and water can be supplied into the tank 72.

(8) According to the first embodiment, the heater 71 is arranged below the water storage space WS. Water stored in the water storage space WS is positioned on a lower side in the vertical direction in accordance with gravity. Thus, when the heater 71 is arranged below the water storage space, the heater 71 is close to the water, thus the water stored in the water storage space WS is easily heated. Further, since the material for the heater contact portion 73D of the bottom wall 73A of the tank 72 facing the heater 71 is metal, heat of the heater 71 can be efficiently transferred to the water stored in the water storage space WS.

(9) According to the above first embodiment, the material for the partition wall 74 is resin, and the material for the heater contact portion 73D is metal. That is, the material for the partition wall 74 is a material having lower heat conductivity than that of the material for the bottom wall 73A including the heater contact portion 73D. Thus, heat of water stored in the water storage space WS is less likely to be absorbed by the partition wall 74. As a result, heating efficiency of the humidifier 70 as a whole is improved.

(10) According to the above first embodiment, the trap space TS surrounds an entire periphery of the water storage space from an outside, when viewed from above. When water stored in the water storage space WS is heated, heat of the water storage space WS is released to a circumference, however, since the trap space TS, which is a space as an air layer, increases heat insulating properties, it is easy to confine the heat in the water storage space WS. As a result, heating efficiency of the humidifier 70 as a whole is improved. Further, since the sponge 76 housed in the trap space TS has a relatively large space, it is difficult to inhibit the high heat insulating properties of the trap space TS.

### Second Embodiment

Next, a second embodiment in which a humidifier is applied to a CPAP device will be described. Note that, in the following description of the second embodiment, a configuration similar to that of the first embodiment is assigned the same reference numeral, and a specific description thereof will be omitted or simplified.

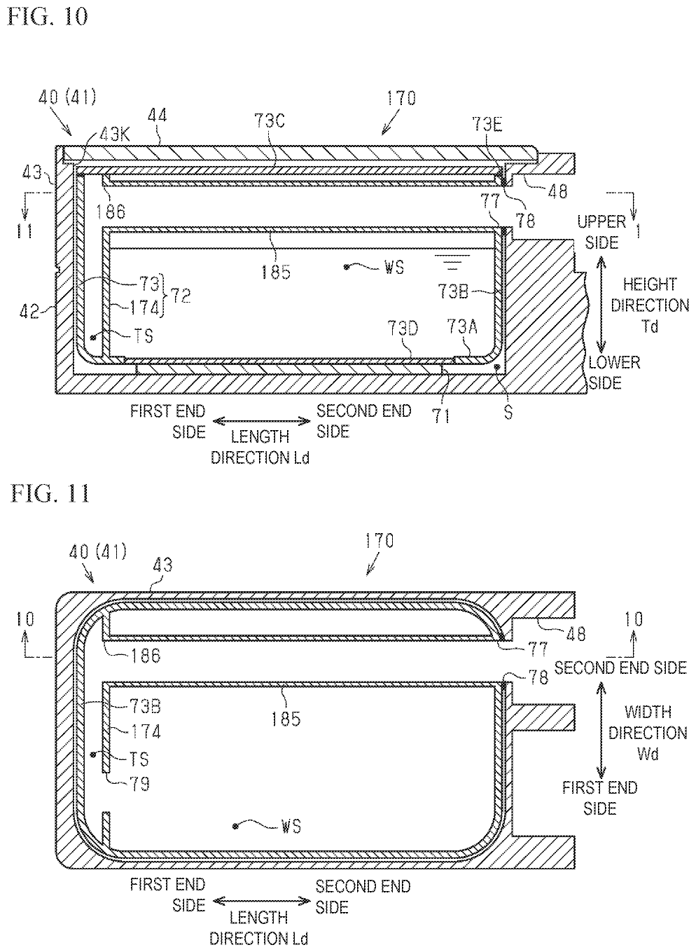

As illustrated in FIG. 10, in a humidifier 170, a partition wall 174 in a rectangular shape in plan view rises from the bottom wall 73A toward the upper wall 73C. The partition wall 174 is positioned closer to a first end side in the length direction Ld than the heater contact portion 73D in the length direction Ld. A dimension in the width direction Wd of the partition wall 174 is a distance between a surface on a first end side and a surface on a second end side of the side wall 73B in the width direction Wd. Then, a dimension in the height direction Td of the partition wall 174 is a distance in the height direction Td from the bottom wall 73A to the upper wall 73C. As a result, an internal space of the tank 72 is partitioned by the partition wall 174 into the trap space TS on the first end side, and the water storage space WS on the second end side in the length direction Ld. Note that, the partition wall 174 is an integrally molded product with the bottom wall 73A and the side wall 73B. Further, the upper wall 73C can be attached to and detached from the side wall 73B and the partition wall 174.

As illustrated in FIG. 11, in addition to the partition wall hole 79, a through-hole 186 in a circular shape in plan view serving as a communication hole opens in the partition wall 174. Additionally, a pipe 185 in a cylindrical shape extends from the through-hole 186 to the first tank hole 77.

Next, an action of the humidifier 170 in the second embodiment will be described.

In a first use state of the CPAP device 10, air flowing from the third introduction hole 48 passes through the pipe 185 via the first tank hole 77, and flows from the through-hole 186 into the trap space TS. Then, the air passes through the partition wall hole 79 from the trap space TS, is humidified in the water storage space WS, and is led out from the third lead-out portion 50.

Next, effects of the humidifier 170 in the above second embodiment will be described. According to the above second embodiment, in addition to the effects (1), (3), (7) to (9) of the above first embodiment, the following effects are exhibited.

(11) According to the above second embodiment, in order to leak from the first tank hole 77, water stored in the water storage space WS needs to pass through the trap space TS arranged between the water storage space WS and the first tank hole 77, and an internal space of the pipe 185. In the above second embodiment, the pipe 185 allows the trap space TS to be arranged on the first end side in the length direction Ld of the water storage space WS. Thus, even when the trap space TS does not cause the water storage space WS and an external space of the first tank hole 77 to directly communicate with each other, it is possible to realize a configuration in which the first tank hole 77 and the partition wall hole 79 open in opposite directions.

Each of the above embodiments can be modified and implemented as follows. Each embodiment, and modifications described below can be combined and implemented within a range where technical inconsistency does not occur.

In each of the above embodiments, the humidifier is not limited to be applied to the CPAP device 10. For example, the humidifier may be applied to a respirator, or the humidifier may be used alone.

In each of the above embodiments, the configuration of the base unit 40 is not limited to the example of each of the above embodiments. For example, the second silencer 51 need not be provided.

In each of the above embodiments, the CPAP device 10 need not be used in the second use state. That is, the CPAP device 10 may be used only in the first use state, and used with the blower 31 constantly fixed to the base unit 40.

In each of the above embodiments, the position of the blower 31 is not limited to the example of each of the above embodiments. For example, a suction unit provided with a blower that sends air to the user 93 may be mounted to the third lead-out portion 50, and the air tube 91 may be mounted to the suction unit. In this case, the blower provided in the suction unit pulls air in the humidifier and sends humidified air to the air tube 91.

In each of the above embodiments, the shape of each of the first housing 21 and the second housing 41 is not limited to the example of each of the embodiments described above. For example, the second housing 41 functioning as a humidifier body may have any shape as long as the tank 72 is attachable to and detachable from the second housing 41, and for example, the tank 72 may be configured to be attachable to and detachable from the second housing 41, by providing a protrusion on an outer surface of the second housing 41, and inserting the protrusion into the tank 72. In this case, the housing space S need not be defined in the second housing 41, and the lid 44 may be omitted.

In each of the above embodiments, the shape of the tank 72 is not limited to the example of each of the above embodiments as long as water can be stored with the shape. For example, the shape may be a cylindrical box shape or a polygonal box shape.

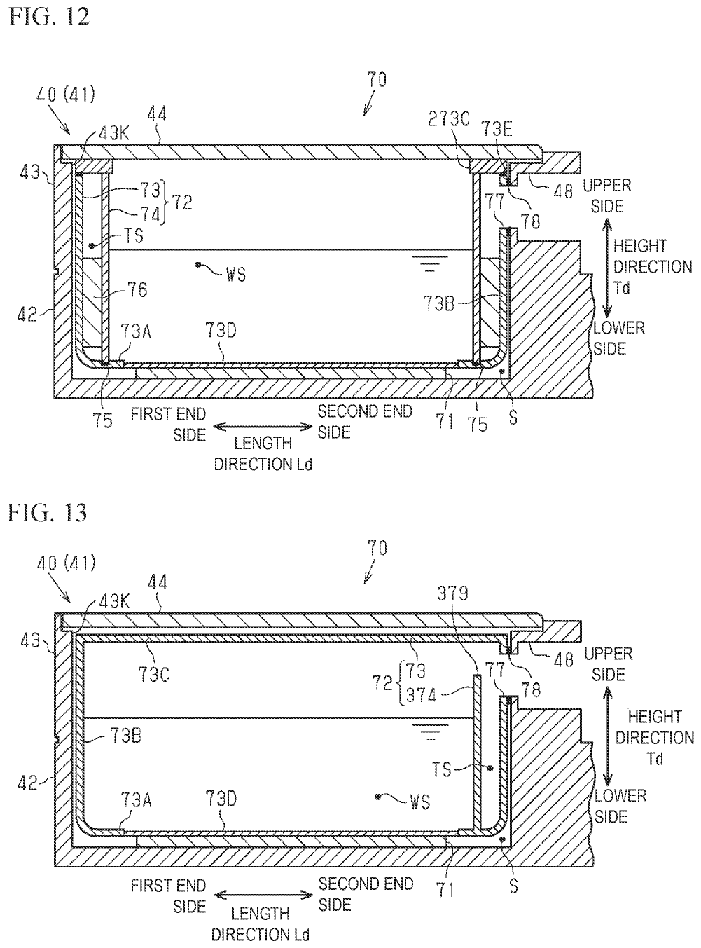

In each of the above embodiments, the shape of the upper wall is not limited to the example of each of the above embodiments. For example, as illustrated in FIG. 12, an upper wall 273C opens an entire upper side in the height direction Td of the water storage space WS, and seals the trap space TS. Specifically, when viewed from the height direction Td, an annular shape is provided that opens at a position of the water storage space WS, and covers a position of the trap space TS. That is, the shape of the upper wall may be changed as appropriate as long as the trap space TS is sealed.

In the above first embodiment, the configuration of the connection portion between the upper wall 73C and the side wall 73B is not limited to the example of each of the embodiments described above. As long as the trap space TS is sealed, the configuration of the seal member 73E may be omitted.

In each of the above embodiments, the portion where the partition wall is mounted is not limited to the example of each of the embodiments described above. For example, in the first embodiment, the lower end of the partition wall 74 may be inclined with respect to the height direction Td from the side wall 73B, and rise upward as a whole.

In each of the above embodiments, the manner in which the heater 71 is mounted to the bottom is not limited to the example of each of the above embodiments. For example, the heater 71 may be mounted to the inside of the bottom wall 73A, or may be mounted to a lower side in the height direction Td of the bottom wall 73A.

In each of the above embodiments, the humidification promoting mechanism is not limited to the heater 71 that heats the tank 72. For example, an ultrasonic generator may be provided as the humidification promoting mechanism. In this case, the ultrasonic generator does not necessarily have to be in contact with the tank 72, and when the ultrasonic generator is installed such that a distance from the ultrasonic generator to water in the tank 72 becomes a desired value, the water in the tank 72 can be efficiently atomized by ultrasonic waves, and vaporization can be promoted.

In each of the above embodiments, the position of the humidification promoting mechanism is not limited to the example of each of the above embodiments. The heater 71 may be arranged away from the bottom wall 73A of the tank 72, or may be arranged on the side wall 73B.

In each of the above embodiments, the sponge 76 functions as the water absorbing member, but the configuration of the water absorbing member is not limited to the example in each of the above embodiments. For example, a water-absorbing polymer as the water absorbing member may be housed in the trap space TS, or the water absorbing member may be housed only in a part of the trap space TS.

In each of the above embodiments, the opening direction of the first tank hole 77 and the partition wall hole 79 when viewed from the inside of the tank body 73 is not limited to the example of each of the above embodiments. For example, in the first embodiment, the partition wall hole 79 may be arranged in a portion of the side wall 73B that is perpendicular to the width direction Wd, and the partition wall hole 79 may open to the first end side in the width direction Wd when viewed from the inside of the tank body 73. Even in this case, the first tank hole 77 and the partition wall hole 79 open in different directions, when viewed from the inside of the tank body 73. Further, when viewed from the inside of the tank body 73, the respective opening directions of the first tank hole 77 and the partition wall hole 79 may be the same.

In each of the above embodiments, the partition wall hole for causing the water storage space WS and the trap space TS to communicate with each other can be omitted. For example, as illustrated in FIG. 13, a partition wall 374 may rise from the bottom wall 73A toward the upper wall 73C, and an upper end of the partition wall 374 may be positioned below the upper wall 73C. In this case, the water storage space WS communicates with the trap space TS through an opening 379 between the upper end of the partition wall 374 and the upper wall 73C.

In each of the above embodiments, the material for the tank body 73 is not limited to the example of each of the above embodiments. For example, in the first embodiment, the bottom wall 73A and the side wall 73B may be configured as an integrally molded product made of metal, and the upper wall 73C and the partition wall 74 may be configured as an integrally molded product made of resin. In this case, the bottom wall 73A and the side wall 73B can be easily manufactured. Further, an entirety of the tank body 73 may be made of the same material.

### REFERENCE SIGNS LIST

10 CPAP DEVICE
20 MAIN BODY UNIT
21 FIRST HOUSING
21A FIRST END SURFACE
21B DENT PORTION
21C DENT SURFACE
21D LOWER SIDE SURFACE
21U UPPER SIDE SURFACE
22 OPERATION UNIT
22A SWITCH
22B SWITCH
23 FIRST INTRODUCTION PORT
24 FILTER
25 FIRST LEAD-OUT PORTION
27 FIRST CONNECTOR
31 BLOWER
32 MAIN FLOW PATH
33 FIRST SILENCER
34 PRESSURE SENSOR
35 FLOW RATE SENSOR
36 TEMPERATURE SENSOR
37 FIRST CONTROL UNIT
38 BATTERY
40 BASE UNIT
41 SECOND HOUSING
42 BASE HOUSING
42U UPPER SIDE SURFACE
43 PROTRUDING HOUSING
43B SECOND END SURFACE
43H FIRST FITTING PORTION
43K ATTACHMENT/DETACHMENT PORT
44 LID
45 PROTRUSION
46 SECOND INTRODUCTION PORT
47 SECOND LEAD-OUT PORT
48 THIRD INTRODUCTION HOLE
49 SECOND CONNECTOR
50 THIRD LEAD-OUT PORTION
50A LID HOLE
51 SECOND SILENCER
53 UPSTREAM SIDE FLOW PATH
54 DOWNSTREAM SIDE FLOW PATH
56 SECOND CONTROL UNIT
60 SEALING MEMBER
61 FIXING PORTION
62 PROTRUDING PORTION
70 HUMIDIFIER
71 HEATER
72 TANK
73 TANK BODY
73A BOTTOM WALL
73B SIDE WALL
73C UPPER WALL
73D HEATER CONTACT PORTION
73E SEAL MEMBER
74 PARTITION WALL
75 RECESSED PORTION
76 SPONGE
77 FIRST TANK HOLE
78 FIRST O-RING
79 PARTITION WALL HOLE
80 SECOND TANK HOLE
81 SECOND O-RING
97 AIR TUBE
92 MASK
93 USER
185 PIPE
186 THROUGH-HOLE
379 OPENING
S HOUSING SPACE
TS TRAP SPACE
WS WATER STORAGE SPACE

1. A humidifier, comprising:
a water storage tank;
a humidifier body to which the water storage tank is detachably mounted; and
a humidification promoting mechanism configured to vaporize water stored in the water storage tank,
in which a gas humidified by the humidification promoting mechanism is discharged from the water storage tank,
wherein the water storage tank includes a tank body having a space therein, and a partition wall rising from an inner surface of the tank body,
the tank body comprises a tank hole that communicates an inside of the tank body with an outside of the tank body,
the partition wall partitions a space inside the tank body into a water storage space configured to store water and a trap space,
the trap space communicates with an outside of the water storage tank via the tank hole, and the water storage space communicates with the trap space.

2. The humidifier according to claim 1,
wherein a water absorbing member that absorbs water is housed in the trap space.

3. The humidifier according to claim 1,
wherein the partition wall comprises a partition wall hole that communicates the water storage space with the trap space, and
the tank hole and the partition wall hole are open in different directions, when the water storage tank is viewed from above.

4. The humidifier according to claim 3,
wherein the partition wall hole and the tank hole are open in opposite directions, when the water storage tank is viewed from above.

5. The humidifier according to claim 1,
wherein the partition wall is attachable to and detachable from the tank body.

6. The humidifier according to claim 1,
wherein the tank body includes a bottom wall, a side wall extending from each side of the bottom wall, and an upper wall connected to the side wall and facing the bottom wall, and
the partition wall is integrally molded to the upper wall.

7. The humidifier according to claim 6,
wherein the upper wall is attachable to and detachable from the side wall, and
the humidifier further comprises a seal member that is mounted to a connection portion between the side wall and the upper wall and that seals the connection portion.

8. The humidifier according to claim 1,
wherein the humidification promoting mechanism is a heater configured to heat the water storage tank, and
the heater is mounted to a bottom portion of the humidifier body.

9. The humidifier according to claim 1,
wherein the humidification promoting mechanism is a heater configured to heat the water storage tank, and
the partition wall comprises a material having lower thermal conductivity than a thermal conductivity of a bottom wall of the tank body.

10. The humidifier according to claim 1,
wherein the trap space surrounds an entire periphery of the water storage space from an outside, when viewed from above.

11. The humidifier according to claim 1,
wherein the partition wall comprises a partition wall hole that communicates the water storage space with the trap space,
the trap space is on an opposite side to the tank hole, with the water storage space interposed therebetween,
the partition wall comprises a communication hole different from the partition wall hole, and
the water storage tank further comprises a pipe in a cylindrical shape extends from the tank hole to the communication hole.
